(19) United States
(12) Patent Application Publication
(10) Pub. No.: US 2022/0332802 A1
(43) Pub. Date: Oct. 20, 2022

(54) METHODS AND COMPOSITIONS FOR TREATMENT OF NEURODEGENERATION

(71) Applicant: Children's Hospital Medical Center, Cincinnati, OH (US)

(72) Inventor: Manoj Pandey, New Richmond, OH (US)

(21) Appl. No.: 17/615,207

(22) PCT Filed: Jun. 5, 2020
(86) PCT No.: PCT/US2020/036235

§ 371 Date: Nov. 30, 2021

Related U.S. Application Data
(60) Provisional application No. 62/857,972, filed on Jun. 6, 2019.
(60) Provisional application No. 62/983,856, filed on Mar. 2, 2020.

(51) Int. Cl. C07K 16/18; A61P 25/16; A61P 25/28

(52) U.S. Cl. CPC C07K 16/18; A61P 25/16; A61P 25/28; C07K 2317/24

(57) ABSTRACT

Disclosed are methods and compositions for the treatment of an individual having neurodegeneration associated with Gaucher's disease and/or different forms of Parkinson's Disease. The disclosed methods and compositions include administering an inhibitor of the C5a pathway to an individual in need thereof, in an amount sufficient to delay onset of and/or alleviate neurodegeneration in the individual having, or suspected of having, Gaucher's disease. Inhibitors of the C5a pathway may include C5a Receptor (C5aR) inhibitors, antagonists of C5aR1, C5aR2, muteins of C5a, and combinations thereof.

### CROSS-REFERENCE TO RELATED APPLICATIONS

This application claims the benefit of, and priority to, U.S. Provisional Application Ser. No. 62/857,972, filed Jun. 6, 2019, and U.S. Provisional Application Ser. No. 62/983,856, filed Mar. 2, 2020, the contents of each are incorporated by reference in their entirety for all purposes.

### BACKGROUND

Gaucher disease (“GD”) is a rare disease with an incidence of about 1 in 60,000 in the general population and 1 in 850 among Ashkenazi Jewish populations with limited treatment options. Worldwide there are about 121,522 Gaucher disease patients and here in the US, approximately 5000 Americans are suffering from this disease. GD results from mutations in the glucocerebrosidase gene GBA1 causing functional disruption of the encoded lysosomal enzyme, acid beta-glucosidase, leading to excess accumulation of glucosylceramide (GC) mainly in macrophages (Mϕs) and elevated plasma level of cytokines and chemokines in human GD patients. lysosomal enzyme glucocerebrosidase (EC 3.2.1.45, GCase)⁵. Acid beta-glucosidase is crucial for the degradation of GC into glucose and ceramide. The excess accumulation of GC in innate and adaptive immune cells within several visceral organs, bone and brain sparks a pro-inflammatory environment resulting in tissue recruitment of several inflammatory immune cells. This pro-inflammatory environment causes tissue damage and promotes clinical GC manifestation.

In addition to the pro-inflammatory environment, some forms of Gaucher disease (GD) are characterized by the completed GBA1 defect and resultant deficiency of glucocerebrosidase (GCase), which lead to the excess brain accumulation of glucosylceramide that fuel neuropathological changes and spark brain inflammation and neurodegeneration in GD.

Improved treatments are needed. Currently, the cost to treat an individual with enzyme replacement therapy is significant, in the range of approximately S100,000 to S300,000 per year. Similarly, substrate reduction therapy (e.g., eligustat and miglustat) is equally expensive. While alternative treatments have potential, such as gene therapy, substrate reduction therapy, and alternative enzyme replacement products, such treatments have been hampered by limitations in the understanding of disease pathogenesis and toxicity concerns due to the blood brain barrier and procedural risks (particularly with respect to gene therapy methods).

Likewise, approximately 60,000 Americans are diagnosed with PD each year. More than 10 million people worldwide are living with PD. GBA defect and/or GCase deficiency is currently the major risk for developing different forms of PD that include GBA associated PD and sporadic PD. Such PDs are characterized by neuroimmune inflammation and their link to neurodegeneration and the development of PD complications.

Currently, there is need for improved treatment of PD.

Thus, there is an urgent need for alternative therapeutic options for the above-noted disease states and disease states of similar etiology. Further alternative treatments are needed for the management of disease complications in GD, particularly neurodegeneration that and other lysosomal storage diseases associated with increased cellular immune inflammation. The instant disclosure satisfies one or more of these needs in the art.

### BRIEF SUMMARY

Disclosed are methods and compositions for the treatment of an individual having neurodegeneration associated with Gaucher's disease. The disclosed methods and compositions include administering an inhibitor of the C5a-C5aR pathway to an individual in need thereof, in an amount sufficient to delay onset of and/or alleviate neurodegeneration in the individual having, or suspected of having, Gaucher's disease. Inhibitors of the C5a pathway may include C5a Receptor (C5aR) inhibitors, antagonists of C5aR1, C5aR2, muteins of C5a, and combinations thereof.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “antibody” refers to a whole or intact antibody (e.g., IgM, IgG, IgA, IgD, or IgE) molecule that is generated by any one of a variety of methods that are known in the art and described herein. The term “antibody” includes a polyclonal antibody, a monoclonal antibody, a chimerized or chimeric antibody, a humanized antibody, a deimmunized human antibody, and a fully human antibody. The antibody may be made in or derived from any of a variety of species, e.g., mammals such as humans, non-human primates (e.g., monkeys, baboons, or chimpanzees), horses, cattle, pigs, sheep, goats, dogs, cats, rabbits, guinea pigs, gerbils, hamsters, rats, and mice. The antibody may be a purified or a recombinant antibody.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, an “aptamer” refers to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), or facilitating the reaction between the target and another molecule. An aptamer may include any suitable number of nucleotides. Aptamers may be DNA or RNA and may be single stranded, double stranded, or contain double stranded or triple stranded regions.

Percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity may be achieved in various ways that are within the skill in the art, for instance, using publicly available computer software Such as BLAST, BLAST-2, ALIGN, ALIGN-2 or Megalign (DNAS TAR) software. Appropriate parameters for measuring alignment, including any algorithms needed to achieve maximal alignment over the full-length of the sequences being compared may be determined by known methods

As used herein, the term “neurodegeneration” means the progressive loss of neurons. This includes but is not limited to immediate loss of neurons followed by subsequent loss of connecting or adjacent neurons.

As used herein, a subject or individual or patient “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an inhibitor of human complement or an inhibitor of interferon alpha).

As used herein, a subject “at risk for developing” as disease state as described herein is a subject having one or more (e.g., two, three, four, five, six, seven, or eight or more) risk factors for developing the disorder. Risk factors for neurodegenerative disease are well known in the art of medicine and include, e.g., a predisposition to develop the condition, i.e., a family history of the condition or a genetic status associated with Gaucher disease.

A subject “suspected of having a disease” is one having one or more (e.g., two, three, four, five, six, seven, eight, nine, or 10 or more) symptoms of the condition.

“Small molecule” as used herein, is meant to refer to an agent, which has a molecular weight of less than about 6 kDa and most preferably less than about 2.5 kDa. Many pharmaceutical companies have extensive libraries of chemical and/or biological mixtures comprising arrays of small molecules, often fungal, bacterial, or algal extracts, which may be screened with any of the assays of the application. This application contemplates using, among other things, small chemical libraries, peptide libraries, or collections of natural products. Tan et al. described a library with over two million synthetic compounds that is compatible with miniaturized cell-based assays (J Am Chem Soc (1998) 120:8565-8566). It is within the scope of this application that such a library may be used to screen for inhibitors of human complement component C5. There are numerous commercially available compound libraries, such as the Chembridge DIVERSet. Libraries are also available from academic investigators, such as the Diversity set from the NCI developmental therapeutics program. Rational drug design may also be employed. For example, rational drug design may employ the use of crystal or solution structural information on the human complement component C5 protein. See, e.g., the structures described in Hagemann et al. (2008) J Biol Chem 283(12):7763-75 and Zuiderweg et al. (1989) Biochemistry 28(1):172-85. Rational drug design may also be achieved based on known compounds, e.g., a known inhibitor of C5 (e.g., an antibody, or antigen-binding fragment thereof, that binds to a human complement component C5 protein).

Monitoring a subject (e.g., a human patient) for an improvement in a disease, as defined herein, means evaluating the subject for a change in a disease parameter, e.g., an improvement in one or more symptoms of the disease. For example, a medical practitioner may examine the extent of the symptoms or signs associated with the disease before and after treatment using a complement inhibitor described herein. Such symptoms include any of the symptoms of the disease state described herein. In some embodiments, the evaluation is performed at least 1 hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject may be evaluated in one or more of the following periods: prior to beginning of treatment; during the treatment; or after one or more elements of the treatment have been administered. Evaluating may include evaluating the need for further treatment, e.g., evaluating whether a dosage, frequency of administration, or duration of treatment should be altered. It may also include evaluating the need to add or drop a selected therapeutic modality, e.g., adding or dropping any of the treatments for the disease described herein.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein may also be used in the practice or testing of the presently disclosed methods and compositions.

The Complement Pathway

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade progresses via the classical pathway, the alternative pathway, or the lectin pathway. These pathways share many components, and while they differ in their initial steps, they converge and share the same “terminal complement” components (C5 through C9) responsible for the activation and destruction of target cells.

The classical complement pathway is typically initiated by antibody recognition of, and binding to, an antigenic site on a target cell. The alternative pathway may be antibody independent, and may be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease (which is different in each pathway) to yield C3a and C3b. Other pathways activating complement attack may act later in the sequence of events leading to various aspects of complement function.

C3a is an anaphylatoxin. C3b binds to bacterial and other cells, as well as to certain viruses and immune complexes, and tags them for removal from the circulation. (C3b in this role is known as opsonin.) The opsonic function of C3b is generally considered to be the most important anti-infective action of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone.

C3b also forms a complex with other components unique to each pathway to form classical or alternative C5 convertase, which cleaves C5 into C5a and C5b. C3 is thus regarded as the central protein in the complement reaction sequence since it is essential to both the alternative and classical pathways. This property of C3b is regulated by the serum protease Factor I, which acts on C3b to produce iC3b. While still functional as opsonin, iC3b cannot form an active C5 convertase.

C5 is a 190 kDa beta globulin found in normal serum at a concentration of approximately 75 μg/mL (0.4 μM). C5 is glycosylated, with about 1.5 to 3 percent of its mass attributed to carbohydrate. Mature C5 is a heterodimer of a 999 amino acid 115 kDa alpha chain that is disulfide linked to a 655 amino acid 75 kDa beta chain. C5 is synthesized as a single chain precursor protein product of a single copy gene (Haviland et al. (1991) J Immunol 146:362-368). The cDNA sequence of the transcript of this gene predicts a secreted pro-C5 precursor of 1658 amino acids along with an 18 amino acid leader sequence (see, e.g., U.S. Pat. No. 6,355,245).

The pro-C5 precursor is cleaved after amino acids 655 and 659, to yield the beta chain as an amino terminal fragment (amino acid residues+1 to 655 of the above sequence) and the alpha chain as a carboxyl terminal fragment (amino acid residues 660 to 1658 of the above sequence), with four amino acids (amino acid residues 656-659 of the above sequence) deleted between the two.

C5a is cleaved from the alpha chain of C5 by either alternative or classical C5 convertase as an amino terminal fragment comprising the first 74 amino acids of the alpha chain (i e, amino acid residues 660-733 of the above sequence). Approximately 20 percent of the 11 kDa mass of C5a is attributed to carbohydrate. The cleavage site for convertase action is at, or immediately adjacent to, amino acid residue 733 of the above sequence. A compound that would bind at, or adjacent, to this cleavage site would have the potential to block access of the C5 convertase enzymes to the cleavage site and thereby act as a complement inhibitor.

C5 may also be activated by means other than C5 convertase activity. Limited trypsin digestion (see, e.g., Minta and Man (1997) J Immunol 119:1597-1602 and Wetsel and Kolb (1982) J Immunol 128:2209-2216) and acid treatment (Yamamoto and Gewurz (1978) J Immunol 120:2008 and Damerau et al. (1989) Molec Immunol 26:1133-1142) may also cleave C5 and produce active C5b.

Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

C5b combines with C6, C7, and C8 to form the C5b-8 complex at the surface of the target cell. Upon binding of several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs may produce other effects. In particular, membrane insertion of small numbers of the C5b-9 complexes into endothelial cells and platelets may cause deleterious cell activation. In some cases, activation may precede cell lysis.

As mentioned above, C3a and C5a are anaphylatoxins. These activated complement components may trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

C5a receptors are found on the surfaces of bronchial and alveolar epithelial cells and bronchial smooth muscle cells. C5a receptors have also been found on eosinophils, mast cells, monocytes, neutrophils, and activated lymphocytes.

Normal alpha-synuclein (α-syn) is required for several of the neuronal function and their survival. Applicant has found that, in a C5aR1 sufficient Gba1 mouse model of Gaucher disease showed marked reduction in level of healthy alpha-synuclein. However, genetically deficiency or pharmaceutical blockade of C5aR1 in Gba1 mouse model of Gaucher disease showed upregulation of normal alpha-synuclein. These data support that activation of C5a-C5aR1 cascades cause down regulation of healthy alpha-synuclein followed by neurodegeneration. Thus, applicant has discovered that the C5a/C5aR1 axis is a critical driver of neurodegeneration in GD. Likewise, the C5a/C5aR1 axis regulates the blood brain barrier integrity in systemic lupus and promotes neurodegeneration in Alzheimer's disease, such that it is believed that alpha-synuclein accumulation observed in Parkinson's may similarly be reversed via C5aR1 blockade.

The pathology of neurological failings in Gaucher disease (GD) is characterized by alteration in neuronal α-synuclein protein (α-syn), pro-inflammatory cytokines, and neurodegeneration. However, the mechanism, by which such pro-inflammatory environment grows and triggers neurodegeneration in GD is wanted.

Methods

The present disclosure relates to compositions containing an inhibitor of human complement (e.g., an inhibitor of complement component C5 such as an anti-C5 antibody) and methods for using the compositions to treat, prevent, slow the progression of, reverse, abate, or ameliorate alpha-synuclein associated neurodegeneration, in particular, wherein the individual has Gaucher disease. In one aspect, novel treatments of alpha-synuclein associated neurodegeneration in an individual having Gaucher disease using a complement inhibitor is disclosed. While not intending to be limiting, exemplary compositions (e.g., pharmaceutical compositions and formulations) and methods for using the compositions are elaborated on below and exemplified in the Examples.

In some aspects, any of the methods described herein may further include identifying the individual as one having, suspected of having, or at risk for developing, alpha-synuclein associated neurodegeneration, particularly wherein said individual has, or is suspected of having, Gaucher disease. In some embodiments, any of the methods described herein may include, after administering a therapeutic agent as disclosed herein, monitoring the individual for an improvement in one or more symptoms of alpha-synuclein associated neurodegeneration in an individual having Gaucher disease.

In one aspect, the disclosure provides a method for treating afflicted with alpha-synuclein associated neurodegeneration, particularly wherein said individual has, or is suspected of having, Gaucher disease using a complement inhibitor, in an amount sufficient to treat the condition.

In one aspect, the disclosure relates to a method of treating a patient afflicted with alpha-synuclein associated neurodegeneration associated with Gaucher disease, which method includes chronically administering to an individual having alpha-synuclein associated neurodegeneration associated with Gaucher disease a complement inhibitor in an amount and with a frequency sufficient to maintain a reduced level of complement activity in the individual to thereby treat neurodegeneration in an individual having Gaucher disease.

In one aspect, the disclosure relates to a method for treating alpha-synuclein associated neurodegeneration associated with Gaucher disease using a complement inhibitor, comprising identifying an individual as being, or likely to be, afflicted with alpha-synuclein associated neurodegeneration associated with Gaucher disease; and administering to the individual a complement inhibitor in an amount sufficient to treat the disease.

In one aspect, the disclosure relates to a method for treating or preventing (e.g., preventing the occurrence of or preventing the progression of the disorder to a more advanced stage) in an individual in need thereof. The method may include administering to the individual a complement inhibitor in an amount sufficient to treat or prevent the alpha-synuclein associated neurodegeneration associated with Gaucher disease.

Complement Inhibitor

In one aspect, the inhibitor of the C5a pathway may be a C5a Receptor (C5aR) inhibitor. The C5aR inhibitor may be one that inhibits C5aR1, C5aR2, or a combination thereof.

In one aspect, the complement inhibitor inhibits the expression of a human complement component protein. In some embodiments, the complement inhibitor may inhibit the activity of a complement protein such as, but not limited to, complement component C1s, complement component C1r, the C3 convertase, the C5 convertase, or C5b-9.

C5a Muteins

In one aspect, the complement inhibitor may be, e.g., one selected from the group consisting of a polypeptide, a polypeptide analog, a nucleic acid, a nucleic acid analog, and a small molecule.

In one aspect, the composition may comprise a C5a Mutein component. The mutein used may be derived from the natural C5a sequence of mammal and non-mammal species. It can, for instance be of human, porcine, murine, bovine or rat origin. In one embodiment, the mutein may be a mutant protein of the human C5a protein.

In one aspect, the inhibitor may be a C5aR1 and C5aR2 inhibitor that is a mutein of C5a. For example, the mutein may comprise a sequence selected from SEQ ID NOS: 9-18, or a mutein of C5a having sequence identity to SEQ ID NO. 16 (“A8 Δ71-73”), wherein the sequence identity is 95%, 96%, 97%, 98%, 99% or 100%. SEQ ID NO. 16, or “A8Δ71-73” is described in U.S. Pat. No. 8,524,862 issued on Sep. 13, 2013 to Magnus Otto and Jörg Köhl, and is further described in Pandey et al Nature 2017.

The mutein used may comprise or has as C-terminal sequence a sequence selected from the group consisting of 67-FKRSLLR-73 (SEQ ID NO: 1) (cf. mutein ABB; SEQ ID NO: 9), 67-FKRLLLR-73 (SEQ ID NO: 2) (cf. mutein A8B-Leu-70; SEQ ID NO: 10), 67-FKRSC-71 (SEQ ID NO: 3) (cf. mutein Ab8-Cys71, SEQ ID NO: 11), 67-FKRSLLC-73 (SEQ ID NO: 4) (cf. mutein Ab8-Cys73, SEQ ID NO: 12), 67-FKRLLLY-73 (SEQ ID NO: 5) (cf. mutein A8B-Leu70-Tyr73, SEQ ID NO: 13), 67-FKKALLR-73 (SEQ ID NO: 6) (cf. mutein A8B-Lys69Ala70; SEQ ID NO: 14), 67-FKRS-70 (SEQ ID NO: 7) (cf. A8B-Del.71-73, SEQ ID NO: 16) and 67-FKLLLLR-73 (cf. A5a, SEQ ID NO: 18).

For the sake of clarity, the numbering refers to the amino acid position of C5a, i.e., 67-F means that phenylalanine is present as amino acid at sequence position 67.

The mutein may further comprise an Arg residue at sequence position 27, see, for example mutein C5a-(1-66, Cys27Arg)-FKRSLLR (A8B-Arg27, SEQ ID NO: 15).

In fact, Arg at position 27 is found in porcine and bovine C5a. In addition, muteins of human C5a with a Cys27Arg replacement were selected from C5a mutant phage library (Cain, S., et al. “Analysis of receptor/ligand interactions using whole-molecule randomly-mutated ligand libraries,” J. Immunol. Methods. 2000. pp 139-145, 245), incorporated herein by reference. Muteins of C5a with only a Cys27Arg replacement are agonists of the C5a receptor (Ibid.).

Exemplary muteins of the human C5a anaphylatoxin having or comprising the amino acid sequence include

SEQ ID NO: 9, i.e. C5a-(1-66, Cys27Ala-)A8B;

SEQ ID NO: 10, i.e., C5a-(1-66, Cys27Ala)-A8B-Leu 70;

SEQ ID NO: 11, i.e., C5a-(1-66, Cys27Ala)-A8B-Cys71;

SEQ ID NO: 12, i.e., C5a-(1-66, Cys27Ala)-A8B-Cys73;

SEQ ID NO: 13, i.e., C5a-(1-66, Cys27Ala)-A8B-Leu70-Tyr73);

SEQ ID NO: 14, i.e., C5a-(1-66, Cys27Ala)-A8B-Lys69-Ala70);

SEQ ID NO: 15; i.e., C5a-(1-66, Cys27Arg)-A8B;

SEQ ID NO: 16, i.e., C5a-(1-66, Cys27Ala)-A8B-Del.71-73);

SEQ ID NO: 17, i.e., C5a-(1-66, Cys-3, Gly-2,-1, Cys27Ala)-A8B; and

SEQ ID NO: 18, i.e., C5a-(1-66, Cys27Ala)A5a.

In one aspect, the mutein may comprise a terminal sequence selected from SEQ ID NO: 1; SEQ ID NO: 2, SEQ ID NO: 3, SEQ ID NO: 4, SEQ ID NO: 5, SEQ ID NO: 6, SEQ ID NO: 7, and SEQ ID NO: 8.

In one aspect, the positively charged amino acid residue at sequence position 69 of the C5a mutein is Arg or Lys.

In a further aspect used in the invention, the mutein comprises a hydrophobic amino acid residue at sequence position 67. The aromatic hydrophobic amino acids Trp, Phe and Tyr are particularly preferred as residues at sequence position 67.

Also preferred antagonists are muteins which comprise a hydrophobic amino acid residue at one or more of the sequence positions 70, 71 or 72. Such hydrophobic amino acid residues may be selected independently from each other, they may be identical or different. Preferred hydrophobic residues are Leu, Ile and Ala.

Such muteins preferably comprise, at sequence position 70. an amino acid residue which is selected from Ala or Leu. Other preferred muteins comprise Ser at sequence position 70.

A preferred amino acid at sequence position 71 is Leu. The antagonistic mutein disclosed may also preferably comprise a Leu residue at sequence position 72.

In one aspect, the mutein comprises Leu at all of the sequence positions 70, 71, and 72.

If present, i.e., not deleted, in the C5a mutant, the sequence position 73 is preferably occupied by a Cys, Tyr, Arg or Ser residue. In some aspects, the mutein has a length of 70, 71, 72 or 73 amino acid residues. Arg, Cys, Tyr or Ser may be C-terminal amino acid residues of a truncated mutein, i.e., a mutein having 70, 71, 72, or 73 amino acid residues.

Further, muteins are also within the scope of the disclosure in which the positively charged amino acid at position 69 is the C-terminal (last) residue. Accordingly, such muteins may have a length of 69 amino acids. However, it is also possible to introduce deletions, for example, into the N-terminal region of the protein so that an antagonistic protein used herein may comprise fewer than 69 amino acid residues. For clarity reasons it is noted once again that such deletions can, of course, also be present in muteins of the invention in which residues at sequence positions 70 to 74 are not or only partly deleted.

A mutant C5a antagonist cannot only be present as the isolated (recombinant) protein but it may also be modified. In one aspect, a mutein of the invention may be dimerized either with the same or a different mutein to form a homo- or heterodimer. For this purpose, the mutein may comprise an N-terminal linker sequence which is capable of dimerizing the C5a mutein. One example of a preferred linker sequence linked to the N-terminus comprises the sequence Cys-Gly-Gly which may be used for spontaneous dimerization of the C5a mutein A8B in the course of the recombinant production of the mutant protein. Another example of such a suitable linker is Cys-(Gly-Gly-Gly-Gly-Ser)2 (SEQ ID NO: 19).

If the mutein carries a cysteine as C-terminal residue (cf. the muteins A813-Cys71 and A8B-Cys73), the dimerization may also occur by coupling of two muteins via these C-terminal cysteine residues as described by Pellas et al., “Novel C5a receptor antagonists regulate neutrophil functions in vitro and in vivo,” Journal of Immunology, Jun. 1, 1998, pp 5616-5621, vol. 160, no. 11, incorporated herein by reference. The dimerization may also be achieved by linking a nucleotide sequence encoding a mutein in an appropriate reading frame with the nucleotide sequence coding for a protein which forms a homodimer in its native fold. Subsequent expression of the nucleic acid molecule yields a fusion protein consisting of the dimerization module linked to the C5a mutant polypeptide, which then dimerizes spontaneously. Examples of such proteins which may be used as dimerization modules are alkaline phosphatase, superoxide-dismutase or glutathione-S-transferase. The use these proteins is in particular useful because the respective functional fusion protein may readily be obtained by periplasmic expression in bacterial expression systems such as E. coli. The use of dimerization modules such as alkaline phosphatase or superoxide-dismutase provides the further advantage that such a fusion protein may easily be detected using a chromogenic reaction which is catalyzed, e.g., by alkaline phosphatase. Suitable chromogenic substrates for these enzymes, such as 5-bromo-4-chloro-3-indolylphosphate for alkaline phosphatase, are well known to the person skilled in the art. Those fusion proteins are therefore suitable as diagnostic reagents.

In accordance with the disclosure of the above paragraph, the mutein of the invention is in a further aspect linked to a protein or a peptide tag, i.e., in which a fusion protein containing the C5a mutein is also part of the invention. However, the fusion proteins of the mutein A8B with Jun/Fos alone, and, with Jun/Fos and the minor coat protein (pIII) of the filamentous M13 phage fused to the N-terminus of the mutein A8B, which are known from Heller et al, “Selection of a C5a receptor antagonist from phage libraries attenuating the inflammatory response in immune complex disease and ischemia/reperfusion injury,” Journal of Immunology, Jul. 15, 1999, pp 985-994, vol. 163, no. 2, are excluded from the invention. The same applies to the mutein A8B that has a hexahistidine tag directly fused to the N-terminus, because this polypeptide is known from Hennecke, Untersuchung zur C5a-C5a Rezeptor-Interaktion unter Verwendung des Phage-Displays, PhD thesis, 1998, Medical School Hannover, Germany.

A fusion protein of the invention may comprise any suitable fusion partner, e.g., alkaline phosphatase or the green fluorescent protein (GFP) as long as the fusion partner does not interfere with the antagonistic properties of the mutein disclosed here and converts the mutein into an agonist when given to a patient, for example. A fusion partner appropriate for therapeutic purpose is a protein such as albumin which may enhance the in vivo (circulation) half-life of a mutein of the invention. The fusion partner may be fused to the N-terminus of the C5a mutein. Likewise, any peptide tag may be fused to the N-terminus of the mutein as long as its antagonistic property is maintained. Examples of suitable affinity tags are the STREP-TAG® which has specific binding affinity for streptavidin or mutants thereof as STREP-TACTIN® (see U.S. Pat. No. 5,506,121, Skerra et al., issued Apr. 9, 1996, and U.S. Pat. No. 6,022,951, Sano et al., issued Feb. 8, 2000, both incorporated by reference herein), the Flag-tag or the myc-tag, all of which may be used for purification of the mutein by affinity chromatography.

It should, however, be noted that in the event of, e.g., inventive C5a muteins conjugated or fused to a partner that confers agonistic properties, the antagonistic muteins may be readily generated/released from its (fusion) partner by treatment such as limited proteolysis or cleavage, for example enzymatic or chemical cleavage, of a (peptide) bond which links the C5a mutein to the (fusion) partner. Accordingly, it is also within the scope of the present invention, to use a fusion partner, for example, for improved purification of the mutein, for example, even if this fusion partner confers an agonistic activity as long as this activity may be eliminated before (and thus the antagonistic activity of the inventive mutein is generated) the muteins is used, for instance, in a desired therapeutic application. It is also possible to use a mutein the antagonistic activity of which is reduced by the (fusion) partner but not completely abolished. In this case, it is thus not necessary to deliberate the mutein of the invention by cleavage from its (fusion) partner. Rather, the fusion protein or the conjugate as explained in the following may be used in a desired application.

The mutein used in the present invention may also be conjugated to a protein or a different chemical (macromolecular) moiety via a suitable peptidic or non-peptidic linker that may be attached to any suitable residue within the primary sequence of the mutein. A protein can, for instance, be conjugated with the C5a mutein using solvent exposed α-amino groups of lysine residues and glutaraldehyde as linker. Another suitable coupling chemistry is amine-amine crosslinking using bis(succinimidylesters) of 5,5′-dithiobis-(2-nitrobenzoic acid) (DTNB) as described in Haugland, R. Handbook of Fluorescent Probes and Research Chemicals, 6th Ed. 1996, Molecular Probes, Eugene, Oreg., on page 96, incorporated herein by reference. Any protein may be coupled to the C5a mutein, depending on the desired application. For example, a conjugate with streptavidin, horseradish peroxidase or green fluorescent protein might be used as a diagnostic reagent or research tool for visualizing a C5a receptor on the surface or within different compartments of a cell.

Aptamer, Antibody and Small Molecule C5a Pathway Inhibitors

In one aspect, the inhibitor of the C5a pathway may be selected from an aptamer capable of inhibiting the C5a pathway, an anti-C5 antibody such as Eculizumab® (available from Alexion), coversin (a complement inhibitor that acts on complement component-C5, preventing release of C5a and formation of C5b-9, available from Akari Therapeutics and described in Hawksworth et al. Mol Immunol 2017, avacopan (“CCX168,” a small molecule C5aR1 antagonist, Jayne et al. JASN 2017, having the structure

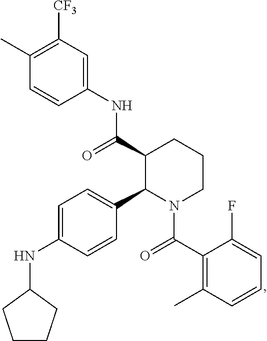 [SMILES: Cc1ccc(NC(=O)[C@H]2CCCN(C(=O)c3c(C)cccc3F)[C@H]2c2ccc(NC3CCCC3)cc2)cc1C(F)(F)F]

or pharmaceutically acceptable salt thereof; a C5a C-terminal cyclic peptide PMX 205 (Cyclo(N2-(Oxo-3phenylpropy)-Orn-Pro-D-Cha-Trp-Arg, (as described in Kumar et al. Sci Reports 2018, Seow et al Sci Rep 2016, Paczkowski et al Br J Pharmacol 1999, Köhl et al. Curr Opin Mol Therap 2007, available from Tocris) having the structure Cyclo[N2-(1-Oxo-3-phenylpropyl)-Orn-Pro-D-Cha-Trp-Arg]), C5aR 1 antagonist, for example, PMX53, available at Calbiochem, having the structure

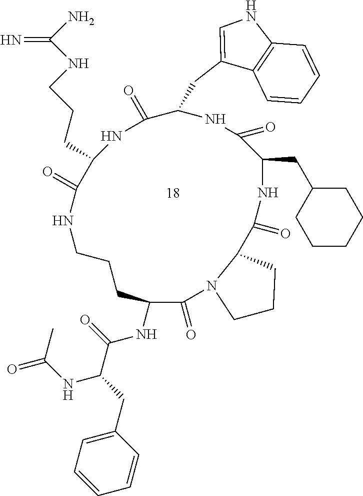 [SMILES: CC(=O)N[C@@H](Cc1ccccc1)C(=O)N[C@H]1CCCNC(=O)[C@H](CCCNC(=N)N)NC(=O)[C@H](Cc2c[nH]c3ccccc23)NC(=O)[C@@H](CC2CCCCC2)NC(=O)[C@@H]2CCCN2C1=O]

or a pharmaceutically acceptable salt thereof.

In one aspect, the inhibitor of the C5a pathway may be eculizumab, wherein the eculizumab may be dosed based on the age of the patient. Patients with 18 years of age and older may require intravenous administration of 600 mg weekly for the first 4 weeks, followed by 900 mg for the fifth dose 1 week later, then 900 mg every 2 weeks thereafter.

In one aspect, the complement inhibitor may be, e.g., one selected from the group consisting of soluble CR1, LEX-CR1, MCP, DAF, CD59, Factor H, cobravenom factor, FUT-175, complestatin, and K76 COOH.

The compositions and methods described herein may include an inhibitor of human complement. Any compound which binds to or otherwise blocks the generation and/or activity of any of the human complement components may be utilized in accordance with the present disclosure. For example, an inhibitor of complement may be, e.g., a small molecule, a nucleic acid or nucleic acid analog, a peptidomimetic, or a macromolecule that is not a nucleic acid or a protein. These agents include, but are not limited to, small organic molecules, RNA aptamers, L-RNA aptamers, Spiegelmers, antisense compounds, double stranded RNA, small interfering RNA, locked nucleic acid inhibitors, and peptide nucleic acid inhibitors. In some embodiments, a complement inhibitor may be a protein or protein fragment.

In some embodiments, the compositions and methods described herein may include antibodies specific to a human complement component. Some compounds include antibodies directed against complement components C1, C2, C3, C4, C5 (or a fragment thereof; see below), C6, C7, C8, C9, Factor D, Factor B, Factor P, MBL, MASP-1, or MASP-2, thus preventing the generation of the anaphylatoxic activity associated with C5a and/or preventing the assembly of the membrane attack complex (MAC) associated with C5b. In some embodiments, the inhibitor of complement inhibits the activity and/or assembly of the C5b-9 complex. For example, in some embodiments, the inhibitor is an antibody or an antigen-binding fragment thereof that binds to one of C6, C7, C8, C9, or C5b to thus prevent the assembly and/or activity of the MAC.

The compositions may also contain naturally occurring or soluble forms of complement inhibitory compounds such as CR1, LEX-CR1, MCP, DAF, CD59, Factor H, cobra venom factor, FUT-175, complestatin, and K76 COOH. Other compounds which may be utilized to bind to or otherwise block the generation and/or activity of any of the human complement components include, but are not limited to, proteins, protein fragments, peptides, small molecules, RNA aptamers including ARC 187 (which is commercially available from Archemix Corporation, Cambridge, Mass.), L-RNA aptamers, spiegelmers, antisense compounds, serine protease inhibitors, molecules which may be utilized in RNA interference (RNAi) such as double stranded RNA including small interfering RNA (siRNA), locked nucleic acid (LNA) inhibitors, peptide nucleic acid (PNA) inhibitors, etc.

In some embodiments, the complement inhibitor inhibits the activation of complement. For example, the complement inhibitor may bind to and inhibit the complement activation activity of C1 (e.g., C1q, C1r, or C1s) or the complement inhibitor may bind to and inhibit (e.g., inhibit cleavage of) C2, C3, or C4. In some embodiments, the inhibitor inhibits formation or assembly of the C3 convertase and/or C5 convertase of the alternative and/or classical pathways of complement. In some embodiments, the complement inhibitor inhibits terminal complement formation, e.g., formation of the C5b-9 membrane attack complex. For example, an antibody complement inhibitor may include an anti-C5 antibody. Such anti-C5 antibodies may directly interact with C5 and/or C5b, so as to inhibit the formation of and/or physiologic function of C5b.

In some embodiments, the compositions described herein may contain an inhibitor of human complement component C5 (e.g., an antibody, or antigen-binding fragment thereof, that binds to a human complement component C5 protein or a biologically-active fragment thereof such as C5a or C5b). As used herein, an “inhibitor of complement component C5” is any agent that inhibits: (i) the expression, or proper intracellular trafficking or secretion by a cell, of a complement component C5 protein; (ii) the activity of C5 cleavage fragments C5a or C5b (e.g., the binding of C5a to its cognate cellular receptors or the binding of C5b to C6 and/or other components of the terminal complement complex; see above); (iii) the cleavage of a human C5 protein to form C5a and C5b; or (iv) the proper intracellular trafficking of, or secretion by a cell, of a complement component C5 protein. Inhibition of complement component C5 protein expression includes: inhibition of transcription of a gene encoding a human C5 protein; increased degradation of an mRNA encoding a human C5 protein; inhibition of translation of an mRNA encoding a human C5 protein; increased degradation of a human C5 protein; inhibition of proper processing of a pre-pro human C5 protein; or inhibition of proper trafficking or secretion by a cell of a human C5 protein. Methods for determining whether a candidate agent is an inhibitor of human complement component C5 are known in the art and described herein.

An inhibitor of human complement component C5 may be, e.g., a small molecule, a polypeptide, a polypeptide analog, a nucleic acid, or a nucleic acid analog.

Peptidomimetics may be compounds in which at least a portion of a subject polypeptide is modified, and the three-dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

Nucleic acid inhibitors may be used to decrease expression of an endogenous gene, e.g., a gene encoding human complement component C5. The nucleic acid antagonist may be, e.g., an siRNA, a dsRNA, a ribozyme, a triple-helix former, an aptamer, or an antisense nucleic acid. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. The siRNA sequences may be, in some embodiments, exactly complementary to the target mRNA. dsRNAs and siRNAs in particular may be used to silence gene expression in mammalian cells (e.g., human cells). See, e.g., Clemens et al. (2000) Proc Natl Acad Sci USA 97:6499-6503; Billy et al. (2001) Proc Natl Acad Sci USA 98:14428-14433; Elbashir et al. (2001) Nature 411:494-8; Yang et al. (2002) Proc Natl Acad Sci USA 99:9942-9947, and U.S. Patent Application Publication Nos. 20030166282, 20030143204, 20040038278, and 20030224432. Anti-sense agents may include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds may include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted. Hybridization of antisense oligonucleotides with mRNA (e.g., an mRNA encoding a human C5 protein) may interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA. Exemplary antisense compounds include DNA or RNA sequences that specifically hybridize to the target nucleic acid, e.g., the mRNA encoding a human complement component C5 protein. The complementary region may extend for between about 8 to about 80 nucleobases. The compounds may include one or more modified nucleobases.

Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include, e.g., 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. See, e.g., U.S. Pat. Nos. 4,987,071; 5,116,742; and U.S. Pat. No. 5,093,246; “Antisense RNA and DNA,” D. A. Melton, Ed., Cold Spring Harbor Laboratory, Cold Spring Harbor, N.Y. (1988); Haselhoff and Gerlach (1988) Nature 334:585-59; Helene, C. (1991) Anticancer Drug D 6:569-84; Helene (1992) Ann NY Acad Sci 660:27-36; and Maher (1992) Bioassays 14:807-15.

Aptamers are short oligonucleotide sequences that may be used to recognize and specifically bind almost any molecule, including cell surface proteins. The systematic evolution of ligands by exponential enrichment (SELEX) process is powerful and may be used to readily identify such aptamers. Aptamers may be made for a wide range of proteins of importance for therapy and diagnostics, such as growth factors and cell surface antigens. These oligonucleotides bind their targets with similar affinities and specificities as antibodies do. See, e.g., Ulrich (2006) Handb Exp Pharmacol 173:305-326.

In some embodiments, the inhibitor of human C5 is an antibody, or antigen-binding fragment thereof, which binds to a human complement component C5 protein. (Hereinafter, the antibody may sometimes be referred to as an “anti-C5 antibody.”)

In some embodiments, the anti-C5 antibody binds to an epitope in the human pro-C5 precursor protein. For example, the anti-C5 antibody may bind to an epitope in the human complement component C5 protein comprising, or consisting of, the amino acid sequence depicted in SEQ ID NO:1 (NCBI Accession No. AAA51925 and Haviland et al., supra).

An “epitope” refers to the site on a protein (e.g., a human complement component C5 protein) that is bound by an antibody. “Overlapping epitopes” include at least one (e.g., two, three, four, five, or six) common amino acid residue(s).

In some embodiments, the anti-C5 antibody binds to an epitope in the human pro-C5 precursor protein lacking the leader sequence. For example, the anti-C5 antibody may bind to an epitope in the human complement component C5 protein comprising, or consisting of, the amino acid sequence depicted in SEQ ID NO:2, which is a human C5 protein lacking the amino terminal leader sequence.

In some embodiments, the anti-C5 antibody may bind to an epitope in the alpha chain of the human complement component C5 protein. For example, the anti-C5 antibody may bind to an epitope within, or overlapping with, a protein having the amino acid sequence of the human complement component C5 alpha chain protein. Antibodies that bind to the alpha chain of C5 are described in, for example, Ames et al. (1994) J Immunol 152:4572-4581.

In some embodiments, the anti-C5 antibody may bind to an epitope in the beta chain of the human complement component C5 protein. For example, the anti-C5 antibody may bind to an epitope within, or overlapping with, a protein having the amino acid sequence depicted in SEQ ID NO:4, which is the human complement component C5 beta chain protein. Antibodies that bind to the C5 beta chain are described in, e.g., Moongkarndi et al. (1982) Immunobiol 162:397; Moongkarndi et al. (1983) Immunobiol 165:323; and Mollnes et al. (1988) Scand J Immunol 28:307-312. In one aspect, the complement inhibitor inhibits the cleavage of human complement component C5, C4, C3, or C2. For example, a complement inhibitor may inhibit the cleavage of complement component C5 into fragments C5a and C5b. 0020. In some embodiments, the complement inhibitor is an antibody or antigen-binding fragment thereof that binds to a human complement component protein (e.g., a C5 protein). In some embodiments, the antibody or antigen-binding fragment thereof binds to the alpha chain of C5 protein. In some embodiments, the antibody or antigen-binding fragment thereof binds to the beta chain of C5. In some embodiments, the antibody or antigen-binding fragment thereof binds to the alpha chain of human complement component C5, and wherein the antibody (i) inhibits complement activation in a human body fluid, (ii) inhibits the binding of purified human complement component C5 to either human complement component C3b or human complement component C4b, and (iii) does not bind to the human complement activation product free C5a. In some embodiments, the antibody binds to the human complement component C5 protein comprising or consisting of the amino acid sequence depicted in any one of SEQ ID NOS:1-26 as described in US 2015/0174243. In some embodiments, the inhibitor is an antibody or antigen-binding fragment thereof that binds to complement component C5 fragment C5b.

In one aspect, the antibody may be a monoclonal antibody. In some embodiments, the antibody or antigen-binding fragment thereof may be one selected from the group consisting of a humanized antibody, a recombinant antibody, a diabody, a chimerized or chimeric antibody, a deimmunized human antibody, a fully human antibody, a single chain antibody, an Fv fragment, an Fd fragment, an Fab fragment, an Fab′ fragment, and an F(ab′)2 fragment.

In one aspect, the complement inhibitor may be eculizumab or pexelizumab. In one aspect the anti-C5a antibody may be CLS026, available from Alexion. CLS026 is a monoclonal mouse IgG1 antibody specific for a neoepitope on murine C5a. CLS026 was derived using a phage display selection on murine C5a, with negative selection against full length human C5. CLS026 was derived from a phage display library using conventional panning techniques, with negative selection against human C5 and converted to a full-length IgG. This neoepitope specific antibody binds to its target, murine C5a, with single digit nM affinity. See, e.g., Coughlin, Beth et al. “Connecting the innate and adaptive immune responses in mouse choroidal neovascularization via the anaphylatoxin C5a and γδT-cells” Scientific reports vol. 6 23794. 31 Mar. 2016, doi:10.1038/srep23794.

In some embodiments, the compositions and methods described herein may include an antibody, or antigen-binding fragment thereof, that binds to a human complement component C5 protein. In some embodiments, the compositions and methods described herein may include an antibody, or antigen-binding fragment thereof, that binds to human C5 fragment C5a or C5b. In some embodiments, the C5 inhibitor is a small molecule or a nucleic acid such as, e.g., a siRNA or an anti-sense RNA that binds to and promotes inactivation of C5 mRNA in a mammal.

In one aspect, a method for treating or preventing a complement-associated disorder in a human is disclosed. The method may include administering to a human in need thereof a therapeutically effective amount of a composition comprising an inhibitor/antibody that blocks human C5/C5a/−receptors (e.g., C5aR1 and C5aR2). Complement-targeting drugs may include anti-C5 antibody Eculizumab, introduced by Alexion and successfully used for treatment of Paroxsysmal Nocturnal Hemoglobinuria (PNH), also approved for atypical hemolytic uremic syndrome associated resulting from uncontrolled C5 activation most frequently seen in response to factor H deficiency (among others) and in testing for additional indications. Other molecules that target C5 include aptamers or the tick-derived molecule cversin (currently tested in clinical trials and reviewed in Hawksworth et al. Mol Immunol 2017). With respect to C5a or C5aR-targeting, avacopan (CCX168), a small molecule C5aR1 antagonist (Avacopan) that has been successfully used to treat patients with ANCA vasculitis in a phase II study (Jayne et al. JASN 2017) and is now tested in a phase III trial. The C5a C-terminal cyclic peptide PMX 205 (Cyclo(N2-(Oxo-3phenylpropy)-Orn-Pro-D-Cha-Trp-Arg) has been successfully used in multiple models of inflammation including allergic asthma, inflammatory bowel disease, ischemia-reperfusion injury and neurodegenerative diseases, among others (Kumar et al. Sci Reports 2018), and may also be used with the disclosed methods. This peptide specifically targets C5aR1 at nanomolar concentrations and acts in a pseudo-irreversible and insurmountable manner (Seow et al Sci Rep 2016, Paczkowski et al Br J Pharmacol 1999) and has been tested in early Phase I human clinical trials (Köhl et al. Curr Opin Mol Therap 2007). In addition to targeting C5aR1, two C5a-neutralizing monoclonal antibodies are currently in phase II studies (Hawksworth et al Mol Immunol 2017) and may be used with the disclosed methods. C5aR2-specific agonists have been described that can specifically activate C5aR2 (Croker et al Immunol Cell Biol 2016) and may be employed with applicant's methods. The C5a mutein A8Δ71-73 mutein targets C5aR1 and C5aR2 simultaneously, as described in U.S. Pat. No. 8,542,862 issued on Sep. 13, 2013. In a cecal ligation and puncture model of severe sepsis, the targeting of C5aR1 and C5aR2 has been demonstrated to improve mouse survival, showing that inflammatory conditions exist where C5aR1 and C5aR1 act in concert to drive disease and where blockade of both anapyhylatoxin receptors is required for therapeutic success (Rittirsch et al Nat Med 2008). More recently, A8Δ71-73 has been successfully used to increase survival and reverse disease signs in experimental Gaucher disease (Pandey et al Nature 2017).

In one aspect, the disclosure features a method for treating or preventing a complement-associated disorder in a human. The method includes administering to a human in need thereof a therapeutically effective amount of a composition comprising an inhibitor of human complement (e.g., an inhibitor of human complement component C5).

In one aspect, the disclosure features a method for treating or preventing a complement-associated disorder in a human, which method comprises administering to a human in need thereof a composition comprising a therapeutically effective amount of an inhibitor of human complement (e.g., an inhibitor of human complement component C5).

In one aspect, the inhibitor may inhibit the expression of a human complement component C5 protein. The inhibitor may inhibit the protein expression of a human complement component C5 protein or inhibit the expression of an mRNA encoding the protein. In some embodiments of any of the methods described herein, the inhibitor may inhibit the cleavage of human complement component C5 into fragments C5a and C5b. In some embodiments of any of the methods described herein, the inhibitor binds to, and inhibits, one or both of C5a and C5b. The inhibitor may be, e.g., an antibody that binds to C5a or C5b. In some embodiments, the inhibitor is an antibody that binds to C5a, but does not bind to full-length C5. In some embodiments, the inhibitor is an antibody that binds to C5b, but does not bind to full-length C5. In some embodiments, the inhibitor is an antibody that binds to a human C5a protein or a fragment thereof, such as that described in U.S. Pat. No. 9,079,949 “Anti-C5 antibodies having improved pharmacokinetics” granted 2015 July 2006 and/or U.S. Pat. No. 9,447,176 “Methods and compositions for treating complement-associated disorders” granted on 2016 Sep. 20.

In some embodiments of any of the methods described herein, the inhibitor may be selected from the group consisting of a polypeptide, a polypeptide analog, a nucleic acid, a nucleic acid analog, and a small molecule. “Polypeptide.” “peptide.” and “protein” are used interchange ably and mean any peptide-linked chain of amino acids, regardless of length or post-translational modification. The complement component proteins described herein (e.g., complement component C2, C3, C4, or C5 proteins) may contain or be wild-type proteins or may be variants that have not more than 50 (e.g., not more than one, two, three, four, five, six, seven, eight, nine, ten, 12, 15, 20, 25, 30, 35, 40, or 50) conservative amino acid substitutions. Conservative substitutions typically include substitutions within the following groups: glycine and alanine; Valine, isoleucine, and leucine; aspartic acid and glutamic acid; asparagine, glutamine, serine, and threonine; lysine, histidine and arginine; and phenylalanine and tyrosine.

The polypeptide may be, or consist of, an antibody, or antigen-binding fragment thereof, that binds to a human complement component C5 protein such as any of those described herein. In some embodiments, the antibody may bind to the alpha chain of the complement component C5 protein. In some aspects, the antibody may bind to the beta chain of the complement component C5 protein. In some embodiments, the antibody may bind to the alpha chain of human complement component C5, and the antibody may (i) inhibit complement activation in a human body fluid, (ii) inhibit the binding of purified human complement component C5 to either human complement component C3b or human complement component C4b, and/or (iii) not bind to the human complement activation product free C5a (or a combination of any of the foregoing properties). In some embodiments, the antibody may be a monoclonal antibody, a single-chain antibody, a humanized antibody, a fully human antibody, a polyclonal antibody, a recombinant antibody, a diabody, a chimerized or chimeric antibody, a deimmunized human antibody, a fully human antibody, a single chain antibody, an Fv fragment, an Fd fragment, an Fab fragment, an Fab′ fragment, or an F(ab′)2 fragment. In some embodiments, the antibody may be eculizumab or pexelizumab.

In some embodiments of any of the methods described herein, the composition may be intravenously administered to the human.

In some embodiments of any of the methods described herein, the composition may be administered to the subject prior to, during, or following a plasma therapy (e.g., plasma exchange or plasma infusion). In some embodiments, administration of the C5 inhibitor to the subject may alleviate the need for plasma therapy by a patient. For example, in some embodiments, administration (e.g., chronic administration) of the C5 inhibitor to the subject may alleviate or substantially reduce the need for plasma therapy by an individual for at least 2 months (e.g., 3 months, 4 months, 5 months, 6 months, 7 months, 8 months, 9 months, 10 months, 11 months, or 12 months or 1, 2, 3, 4, 5, or 6 years or more). In some embodiments, any of the methods described herein may include administering to the subject one or more additional active agents useful for treating neurodegeneration associated with Gaucher disease.

In yet another aspect, the disclosure features an article of manufacture, which includes (or consists of) a container with a label and a composition containing an inhibitor of human complement (e.g., an inhibitor of human complement component C5). The label indicates that the composition is to be administered to a human having, suspected of having, or at risk for developing, alpha-synuclein associated neurodegeneration in the context of Gaucher disease. The inhibitor may be, e.g., an antibody or antigen-binding fragment thereof that binds to complement component C5 or a fragment thereof such as C5a or C5b. In some embodiments, the article of manufacture may contain one or more additional active agents that are useful for treating or preventing alpha-synuclein associated neurodegeneration in the context of Gaucher disease (e.g., ameliorating one or more symptoms of the disorder). Accordingly, in another aspect, the disclosure features a method for treating alpha-synuclein associated neurodegeneration associated with Gaucher disease, or reducing the occurrence or severity of alpha-synuclein associated neurodegeneration associated with Gaucher disease, in an individual who has, is suspected of having, or at risk of developing alpha-synuclein associated neurodegeneration associated with Gaucher disease. The method includes administering to the patient (being in need thereof) an inhibitor of complement such as an inhibitor of complement component C5 to thereby treat alpha-synuclein associated neurodegeneration associated with Gaucher. The inhibitor may be, e.g., any of the C5 inhibitors described herein. Administration of the C5 inhibitor may reduce the occurrence or severity of alpha-synuclein associated neurodegeneration associated with Gaucher disease.

In one aspect, the method for treating alpha-synuclein associated neurodegeneration in an individual having Gaucher disease, may include chronically administering to the patient in need thereof a complement inhibitor (e.g., a C5 inhibitor such as an anti-C5 antibody) in an amount and with a frequency that are effective to maintain systemic complement inhibition in the patient. As used herein, “chronically administered,” “chronic treatment,” “treating chronically,” or similar grammatical variations thereof refer to a treatment regimen that is employed to maintain a certain threshold concentration of a therapeutic agent in the blood of an individual in order to completely or substantially suppress systemic complement activity in the patient over a prolonged period of time. Accordingly, an individual chronically treated with a complement inhibitor may be treated for a period of time that is greater than or equal to 2 weeks (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks; 1, 2, 3, 4, 5, 6, 7, 8, 9, 10, 11, or 12 months; or 1, 1.5, 2, 2.5, 3, 3.5, 4, 4.5, 5, 5.5, 6, 6.5, 7, 7.5, 8, 8.5, 9, 9.5, 10, 10.5, or 12 years or for the remainder of the patient's life) with the inhibitor in an amount and with a dosing frequency that are sufficient to maintain a concentration of the inhibitor in the patient's blood that inhibits or substantially inhibits systemic complement activity in the patient. In some embodiments, the complement inhibitor may be chronically administered to an individual in need thereof in an amount and with a frequency that are effective to maintain serum hemolytic activity at less than or equal to 20 (e.g., 19, 18, 17, 16, 15, 14, 13, 12, 11, 10, 9, 8, 7, 6, or even below 5) % and to maintain serum hemolytic activity at less than or equal to 20%.

To maintain systemic complement inhibition in a patient, the complement inhibitor may be chronically administered to the patient, e.g., once a week, once every two weeks, twice a week, once a day, once a month, or once every three weeks. In some embodiments of any of the methods described herein, a C5 inhibitor (e.g., an anti-C5 antibody) may be administered to an individual in an amount and with a frequency of administration effective to maintain a concentration of at least 0.7 (e.g., at least 0.8, 0.9, one, two, three, four, five, six, seven, eight, nine, or 10 or more) divalent C5 inhibitor molecule(s) (e.g., a whole anti-C5 antibody such as eculizumab) per every C5 molecule in the patient's blood. “Divalent” or “bivalent,” with respect to a C5 inhibitor, refers to a C5 inhibitor that contains at least two binding sites for a C5 molecule. Where the C5 inhibitor is monovalent (e.g., a single chain anti-C5 antibody or a Fab that binds to C5), the inhibitor may be administered to the patient in an amount and with a frequency that are effective to maintain a concentration of at least 1.5 (e.g., at least 2, 2.5, 3, 3.5, 4, 4.5, or 5 or more) of the monovalent C5 inhibitors per every C5 molecule in the blood. In some embodiments, the monovalent C5 inhibitor may be administered to the patient in an amount and with a frequency that are effective to maintain a ratio of monovalent C5 inhibitor to C5 of at least 2:1 (e.g., at least 3:1, at least 4:1, at least 5:1, or at least 6:1 or more). In some embodiments of any of the methods described herein, a whole (bivalent) anti-C5 antibody is administered to the patient in an amount and with a frequency that are effective to maintain a concentration of at least 40 (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 110, or 120 or more) μg of the antibody per milliliter of the patient's blood. In preferred embodiments, a whole anti-C5 antibody is administered in an amount and with a frequency to maintain the antibody at a concentration of at least 50 μg per milliliter of the patient's blood. In preferred embodiments, a whole anti-C5 antibody (e.g., eculizumab) is administered in an amount and with a frequency to maintain the antibody at a concentration of at least 100 μg per milliliter of the patient's blood. In some embodiments of any of the methods described herein, a monovalent anti-C5 antibody (e.g., a single chain antibody or an Fab fragment) may be administered to the patient in an amount and with a frequency that are effective to maintain a concentration of at least 80 (e.g., 81, 82, 83, 84, 85, 86, 87, 88, 89, 90, 91, 92, 93, 94, 95, 96, 97, 98, 99, 100, 110, 115, 120, 125, 130, 135, 140, 145, 150, 155, 160, 165, or a 170 or more) μg of the antibody per milliliter of the patient's blood.

Compositions

In one aspect, active agents provided herein may be administered in an dosage form selected from intravenous or subcutaneous unit dosage form, oral, parenteral, intravenous, and subcutaneous. In some aspects, active agents provided herein may be formulated into liquid preparations for, e.g., oral administration. Suitable forms include suspensions, syrups, elixirs, and the like. In some aspects, unit dosage forms for oral administration include tablets and capsules. Unit dosage forms configured for administration once a day; however, in certain aspects it may be desirable to configure the unit dosage form for administration twice a day, or more.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, active agents provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubrimayt, inert diluent, surface active or dispersing agent. Molded tablets may be made by molding, in a suitable machine, a mixture of the powdered active agent moistened with an inert liquid diluent.

In some embodiments, each tablet or capsule contains from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some embodiments, tablets or capsules are provided in a range of dosages to permit divided dosages to be administered. A dosage appropriate to the patient and the number of doses to be administered daily may thus be conveniently selected. In certain embodiments two or more of the therapeutic agents may be incorporated to be administered into a single tablet or other dosage form (e.g., in a combination therapy); however, in other embodiments the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragamayth, or alginic acid or salts thereof.

Binders may be used to form a hard tablet. Binders include materials from natural products such as acacia, tragamayth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxymethyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy.

The active ingredients may be prepared for pulmonary delivery in particulate form with an average particle size of from 0.1 um or less to 10 um or more, for example, from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used. Liposomes, microcapsules, microspheres, inclusion complexes, and other types of carriers may also be employed.

Pharmaceutical formulations suitable for use with a nebulizer, either jet or ultrasonic, typically comprise the active agent dissolved or suspended in water at a concentration of about 0.01 or less to 100 mg or more of active agent per mL of solution, for example, from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation may also include a buffer and a simple sugar (e.g., for protein stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device generally comprise a finely divided powder containing the active ingredients suspended in a propellant with the aid of a surfactant. The propellant may include conventional propellants, such as chlorofluorocarbons, hydrochlorofluorocarbons, hydrofluorocarbons, and hydrocarbons. Example propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing active agent, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, for example, from about 5, 10, 15, 20, 25, 30, 35, 40, 45, or 50 wt. % to about 55, 60, 65, 70, 75, 80, 85, or 90 wt. % of the formulation.

In some aspects, an active agent provided herein may be administered by intravenous, parenteral, or other injection, in the form of a pyrogen-free, parenterally acceptable aqueous solution or oleaginous suspension. Suspensions may be formulated according to methods well known in the art using suitable dispersing or wetting agents and suspending agents. The preparation of acceptable aqueous solutions with suitable pH, isotonicity, stability, and the like, is within the skill in the art. In some aspects, a pharmaceutical composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art.

The duration of the injection may be adjusted depending upon various factors, and may comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

In some aspects, active agents provided herein may additionally employ adjunct components conventionally found in pharmaceutical compositions in their art-established fashion and at their art-established levels. Thus, for example, the compositions may contain additional compatible pharmaceutically active materials for combination therapy or may contain materials useful in physically formulating various dosage forms, such as excipients, dyes, thickening agents, stabilizers, preservatives or antioxidants.

In some aspects, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating the disease states described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### EXAMPLES

Gaucher Disease (GD)

Applicant has described immune complexes of GC-specific IgG autoantibodies in experimental and clinical GD, which induced massive complement activation and C5a generation. Applicant further found that C5a-mediated activation of its cognate C5a receptor 1 (C5aR1) tips the balance between GC formation and degradation, thereby fueling excess GC accumulation and inflammation in visceral tissues in experimental and clinical GD.

Examining the production of C5a in the brain of Gba1 D409V/knockout (9V/null) GD-prone mice, Applicant has found that C5a production of the brain of 9V/null mice was markedly elevated, when compared to WT control mice. 9V/null mice suffered from massive accumulation of GC in the brain and loss of neurons. Applicant targeted glucocerebrosidase (GCase) with conduritol B epoxide (CBE) in WT and C5aR1^{-/-} mice. Strikingly, CBE-injected WT mice died within 30 days. In contrast, all C5aR1^{-/-} mice survived the 60-day observation window, were protected from CBE-induced accumulation of GC in the brain, and showed a marked reduction of microglial cell activation and only a minor loss of neurons.

Using a mouse model of GD, Applicant found changed level of α-syn, activated subsets of microglial cells (CD45⁺ CD11b⁺ CD11c⁺ Gr1⁺ CD40⁺), increased recruitment of CD4⁺ T cells, higher production of pro-inflammatory cytokines, (e.g., TNFα, IL1β, and IL6), minor loss of MAP2⁺ primary neurons, and cognitive defects in GD. On the basis of these data, it is believed that, without intending to be limited by theory, continuous processing and presentation of α-synuclein by microglial and T cells result in increased production of the pro-inflammatory cytokines that drive blood brain barrier (BBB) breaching, immune cell recruitment, and neurodegeneration in GD.

To investigate α-syn-mediated increased production of pro-inflammatory cytokines cause BBB disintegration, Applicant performed an adoptive transfer test, (e.g., Gaucher mice CD4⁺T cells→WT mice and Gaucher mice CD4⁺T cells→Gaucher mice). Augmented brain invasion of T cells, elevated level of pro-inflammatory cytokines, and neuronal cells demolition have been observed in Gaucher mice. To justify this in ex vivo condition, FACS sorted microglial and CD4⁺T cells from WT and Gaucher mice brains were co-cultured in presence or absence of α-synuclein and several pro-inflammatory cytokines were measured. These data showed that α-synuclein treated Gaucher mice cells cause marked increased production of pro-inflammatory cytokines. Supernatant obtained from α-synuclein stimulated Gaucher mice cells were used to execute in vitro stimulation of primary neurons and cell death were measured. These data showed that α-syn-mediated increased production of pro-inflammatory cytokines are the basis of neurodegeneration in GD. Blocking T cell infiltration and or cytokine/s signaling may be used for treatment of neuropathic GD and potentially advantageous for neurodegeneration in other lysosomal storage diseases.

GBA1 Associated Parkinson's Disease (GBA1-PD)

Parkinson's disease is believed to be caused by a variety of different factors, including aging, environmental (pesticide, metals, drug addiction), and genetic (PINK1, PARKIN, LRRK2, SNCA, and GBA1 mutations), which can lead to motor symptoms (e.g. tremor, stiffness, bradykinesia, postural instability, balance difficulty, walking problems) and non-motor symptoms (constipation, dementia, depression, mood, vision change, and sleep disturbances). Heterozygous mutations in the GBA1 gene represent the most common genetic risk factor for Parkinson's disease. Brain cells display neuroinflammation in GBA1-PD. Applicant has found that GBA1 defects cause C5a-C5aR1-induced neurodegeneration in Parkinson's disease using a mouse model of PD. In particular, Applicant has found that the C5a-C5aR1 Axis is a critical driver of excess accumulation of GC, pro-inflammatory cytokines, and moderate loss of neurons in CBE-induced partial GCase targeted mouse model of PD and that targeting C5aR1 may slow the progression and/or stop the neurological complications of the disease.

Applicant postulates that immune complexes and/or complement activation alter the blood brain barrier integrity and fuel neuroinflammation and/or neurodegeneration in various conditions or disease states including Ischemia, intracerebral hemorrhage, traumatic brain injury, systemic lupus erythematosus lupus, amyotrophic lateral sclerosis, neuromyelitis optica spectrum, Alzheimer's, Myasthenia gravis, and Huntington's diseases.

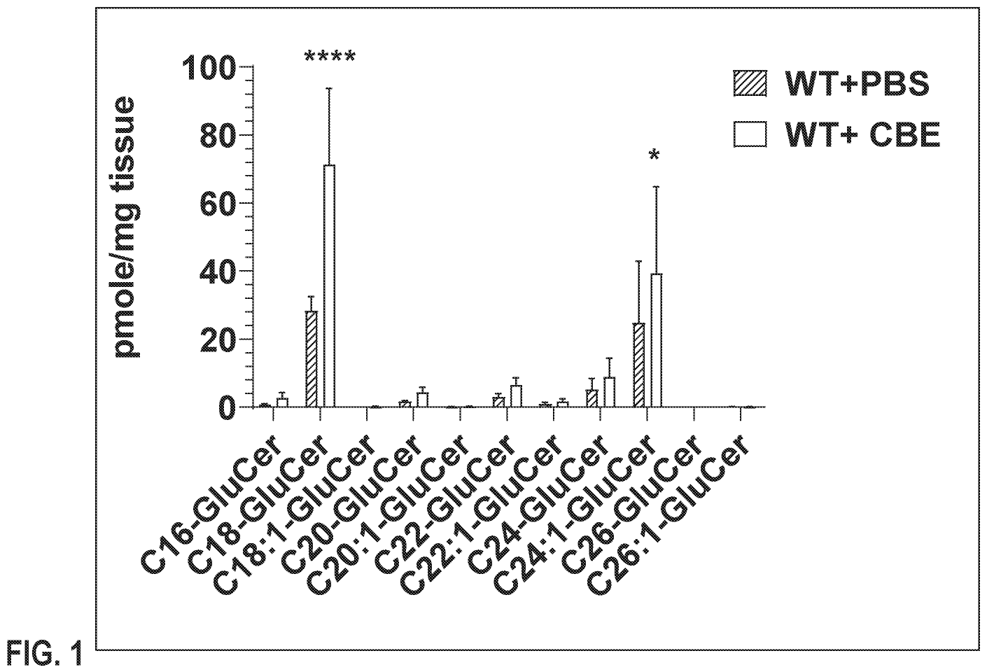

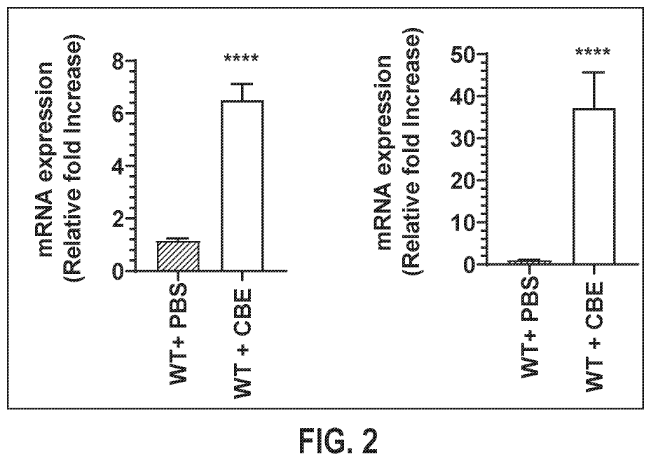

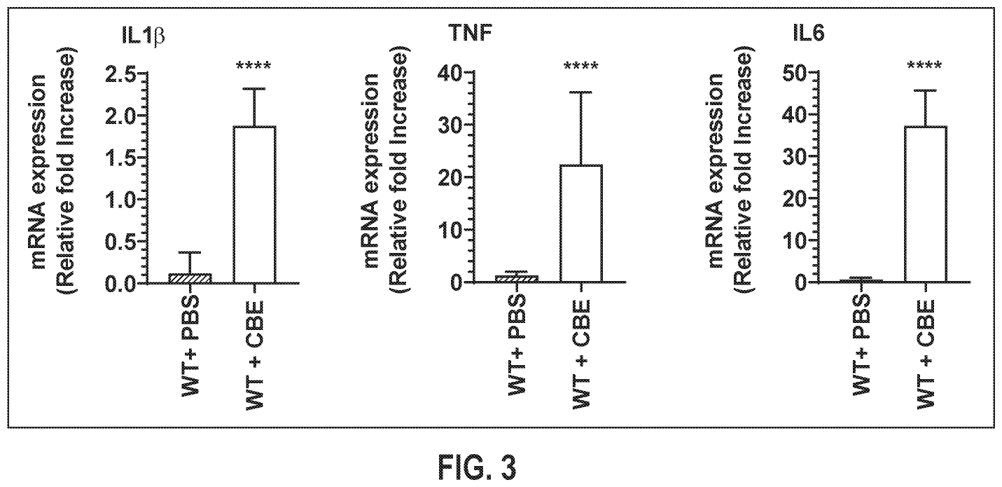

Using a chemically induced GCase-targeted mouse model of Parkinson's disease, as described in Journal of pathology 2016, 239:496-509 (intraperitoneal injection (i.p.) of lower doses of Conduritol B epoxide (50 mg/kg/day for 4 weeks), reduces GCase activity by ˜25-50%, analogous to what is observed in PD with GBA mutations), Applicant demonstrated that the brain from a CBE-GCase targeted PD mouse model showed increased glucosylceramide. (FIG. 1.) Applicant further found that CBE-GCase targeted brains of the PD mouse model have increased C5a and C5aR1 (FIG. 2) and CBE-GCase targeted brains of the PD mouse model have increased pro-inflammatory cytokines (FIG. 3).

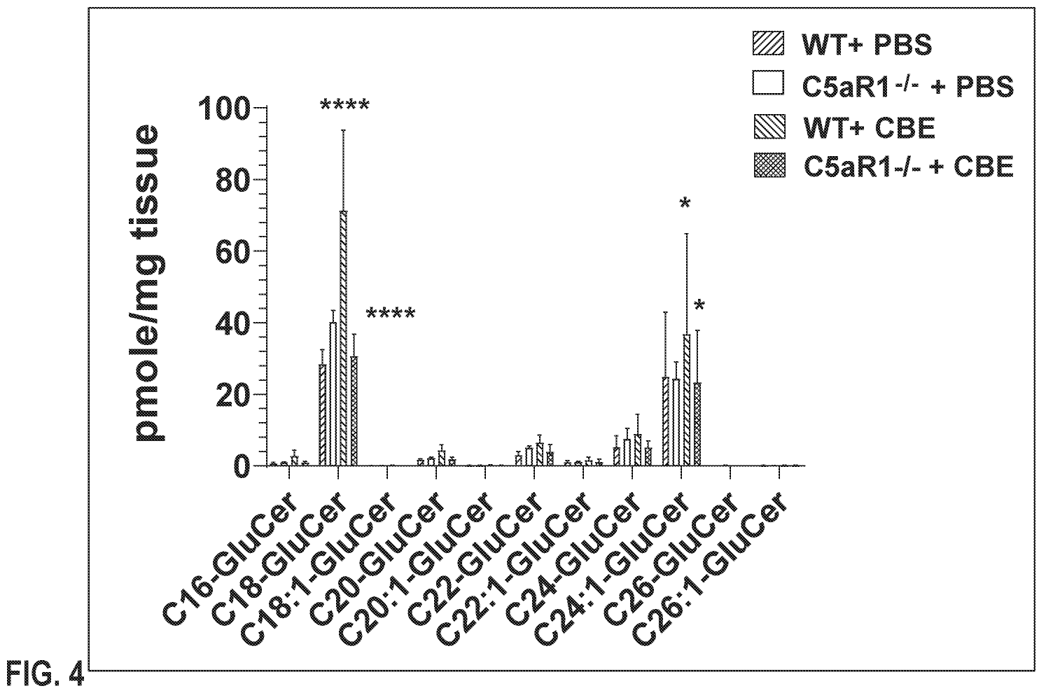

FIG. 4 shows that a genetic deficiency of C5ar1 in a CBE-GCase targeted PD mouse model decreased brain accumulation of GC.

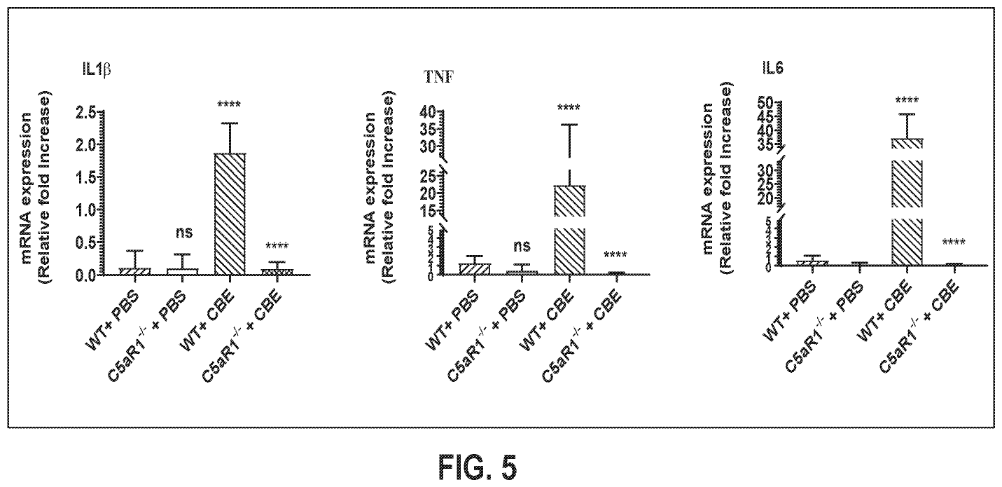

FIG. 5 shows that genetic deficiency of C5ar1 in CBE-GCase targeted PD mouse model showed decreased brain production of pro-inflammatory cytokines.

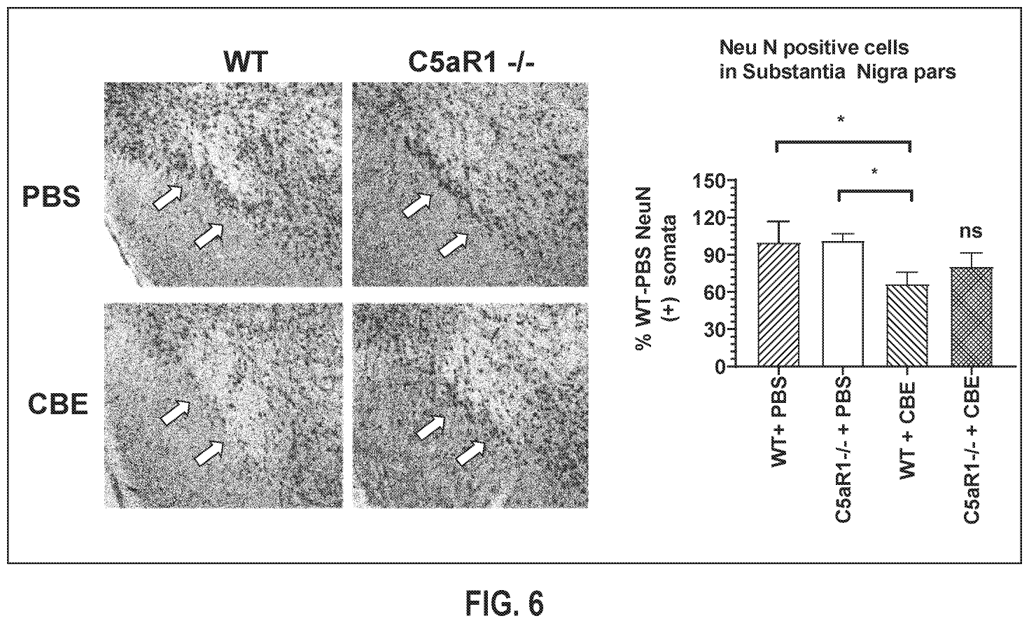

FIG. 6 shows that genetic deficiency of C5aR1 in CBE-GCase targeted PD mouse model showed decreased Loss of NeuN+ Neurons in Substantia Nigra pars compacta.

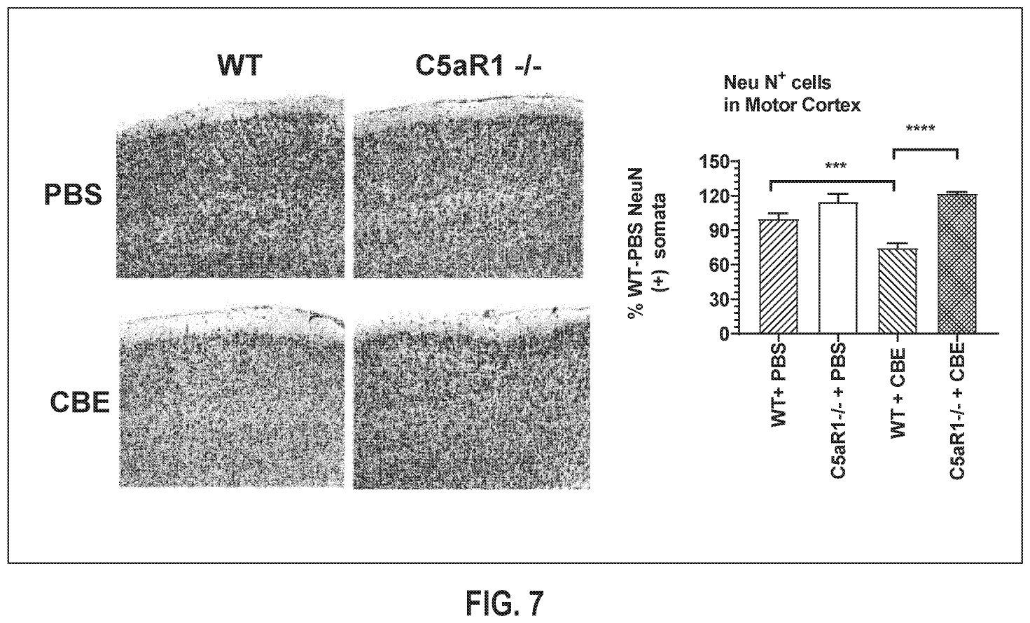

FIG. 7. shows that genetic deficiency of C5ar1 in CBE-GCase targeted PD mouse model showed decreased loss of NeuN+ neurons in motor cortex.

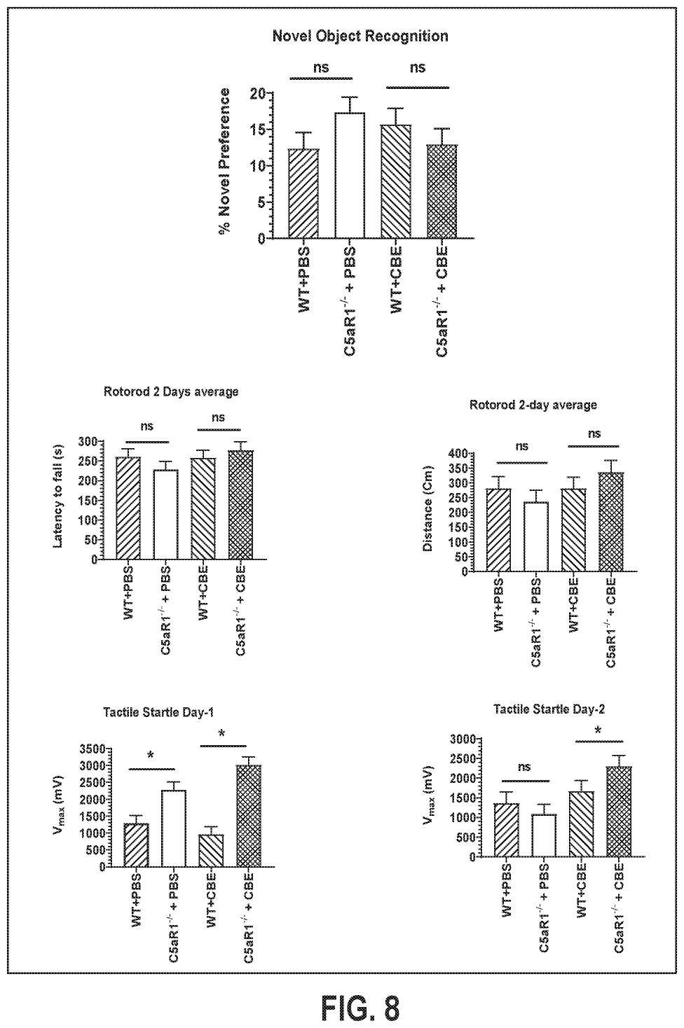

FIG. 8 shows that CBE-GCase targeted WT or C5aR1−/− mice show no motor or memory defects but are hyper-reactive.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1-5. (canceled)

6. A method for treating neurodegeneration in an individual having a disorder selected from one or more of Type I Gaucher disease, Type II Gaucher disease, Type III Gaucher disease, glucocerebrosidase (“GBA”) associated Parkinson's Disease (“PD”), Parkinson's Disease (“PD”) not associated with a GBA mutation or deficiency, Parkinson's Disease (“PD”) with moderate deficiency of GCase, and Alzheimer's disease, comprising the step of administering a C5a inhibitor to said individual.

7. The method of claim 6, wherein said C5a inhibitor is selected from A8^{Δ71-73} and an anti C5 monoclonal antibody, and wherein said C5a inhibitor is administered in an amount sufficient to decrease glucosylceramide accumulation is said individual.

8. The method of claim 6, wherein said Gaucher disease is Type I, Type II, or Type III, and is characterized by a GBA1 mutation.

9. The method of claim 6, wherein said neurodegeneration is associated with increased levels of aggregated alpha-synuclein.

10. The method of claim 6, wherein said C5a inhibitor is an antibody capable of inhibiting the complement pathway.

11. The method of claim 6, wherein said C5a inhibitor is a monoclonal antibody capable of inhibiting the complement pathway.

12. The method of claim 6, wherein said C5a inhibitor is a humanized monoclonal antibody capable of inhibiting the complement pathway.

13. The method of claim 6, wherein said C5a inhibitor is eculizumab.

14. The method of claim 6, wherein said administration step is repeated every day, or every two days, or every three days.

15. The method of claim 6, wherein said administration step is repeated weekly.

16. The method of claim 6, wherein said complement inhibitor is administered from one or more route selected from intravenously and orally.


SEQUENCE LISTING (21 sequences)

SEQ ID NO: 1 - protein, 7 aa, Artificial Sequence
  C5aR Seq 1
FKRSLLR

SEQ ID NO: 2 - protein, 7 aa, Artificial Sequence
  C5aR Seq 2
FKRLLLR

SEQ ID NO: 3 - protein, 5 aa, Artificial Sequence
  C5aR Seq 3
FKRSC

SEQ ID NO: 4 - protein, 7 aa, Artificial Sequence
  C5aR Seq 4
FKRSLLC

SEQ ID NO: 5 - protein, 7 aa, Artificial Sequence
  C5aR Seq 5
FKRLLLY

SEQ ID NO: 6 - protein, 7 aa, Artificial Sequence
  C5aR Seq 6
FKKALLR

SEQ ID NO: 7 - protein, 4 aa, Artificial Sequence
  C5aR Seq 7
FKRS

SEQ ID NO: 8 - protein, 7 aa, Artificial Sequence
  C5aR Seq 8
FKLLLLR

SEQ ID NO: 9 - protein, 73 aa, Homo sapiens
  (C5a-(1-66,Cys27Ala)-A8B
TLQKKIEEIAAKYKHSVVKKCCYDGAAVNNDETCEQRAARISLGPRCIKAFTECCVVASQLRANISFKRSLLR

SEQ ID NO: 10 - protein, 73 aa, Homo sapiens
  (C5A-(1-66,Cys27Ala)-A8B-Leu70
TLQKKIEEIAAKYKHSVVKKCCYDGAAVNNDETCEQRAARISLGPRCIKAFTECCVVASQLRANISFKRLLLR

SEQ ID NO: 11 - protein, 71 aa, Homo sapiens
  (C5a(1-66,Cys27Ala)-A8B-Cys71)
TLQKKIEEIAAKYKHSVVKKCCYDGAAVNNDETCEQRAARISLGPRCIKAFTECCVVASQLRANISFKRSC

SEQ ID NO: 12 - protein, 73 aa, Homo sapiens
  (C5a-(1-66,Cys27Ala)-A8B-Cys73)
TLQKKIEEIAAKYKHSVVKKCCYDGAAVNNDETCEQRAARISLGPRCIKAFTECCVVASQLRANISFKRSLLC

SEQ ID NO: 13 - protein, 73 aa, Homo sapiens
  (C5a(1-66, Cys27Ala)-A8B-Leu70-Tyr73)
TLQKKIEEIAAKYKHSVVKKCCYDGAAVNNDETCEQRAARISLGPRCIKAFTECCVVASQLRANISFKRLLLY

SEQ ID NO: 14 - protein, 73 aa, Homo sapiens
  (C5a-(1-66, Cys27Ala)-A8B-Lys69-Ala70)
TLQKKIEEIAAKYKHSVVKKCCYDGAAVNNDETCEQRAARISLGPRCIKAFTECCVVASQLRANISFKKALLR

SEQ ID NO: 15 - protein, 73 aa, Homo sapiens
  (C5a(1-66,Cys27Arg)-A8b)
TLQKKIEEIAAKYKHSVVKKCCYDGARVNNDETCEQRAARISLGPRCIKAFTECCVVASQLRANISFKRSLLR

SEQ ID NO: 16 - protein, 70 aa, Homo sapiens
  (C5a-(1-66,Cys27Ala)-A8B-Del 71-73
TLQKKIEEIAAKYKHSVVKKCCYDGAAVNNDETCEQRAARISLGPRCIKAFTECCVVASQLRANISFKRS

SEQ ID NO: 17 - protein, 76 aa, Homo sapiens
  (C5a-(1-66,Cys-3,Gly-2,-1,Cys27Ala)-A8B)
CGGTLQKKIEEIAAKYKHSVVKKCCYDGARVNNDETCEQRAARISLGPRCIKAFTECCVVASQLRANISFKRSLLR

SEQ ID NO: 18 - protein, 73 aa, Homo sapiens
  (C5a-(1-66,Cys27Ala)A5a)
TLQKKIEEIAAKYKHSVVKKCCYDGARVNNDETCEQRAARISLGPRCIKAFTECCVVASQLRANISFKLLLLR

SEQ ID NO: 19 - protein, 11 aa, Artificial Sequence
  Linker
CGGGGSGGGGS

SEQ ID NO: 20 - protein, 7 aa, Artificial Sequence
  P32
RHYPYWR

SEQ ID NO: 21 - protein, 6 aa, Artificial Sequence
  P59
LIRLWR